(19) United States
(12) Patent Application Publication
(10) Pub. No.: US 2012/0149125 A1
(43) Pub. Date: Jun. 14, 2012

(54) Ion Population Control for an Electrical Discharge Ionization Source

(76) Inventors: Lee EARLEY, Sunnyvale, CA (US); Christopher Mullen, Menlo Park, CA (US); Jean-Jacques Dunyach, San Jose, CA (US)

(21) Appl. No.: 12/966,956

(22) Filed: Dec. 13, 2010

(51) Int. Cl. G01N 33/00; G05D 23/00; G05D 7/00

(52) U.S. Cl. 436/173; 422/111; 422/109; 422/108

(57) ABSTRACT

A method of providing reagent ions to a mass spectrometer comprises delivering a reagent species to a reagent ionization volume via a passageway at a flow rate. Using previously acquired information, an injection time duration is calculated for injecting reagent ions that are formed in the reagent ionization volume into a reaction region of the mass spectrometer. A determination is made as to whether the calculated injection time duration is within a specified range of injection time duration values. When it is determined that the calculated injection time duration falls outside of the specified range of injection time duration values, the flow rate at which the reagent species is delivered to the ionization volume is adjusted.

### FIELD OF THE INVENTION

The instant invention relates generally to an ionization source for a mass spectrometry system, and more particularly to an ion population control method and system for use with an electrical discharge ionization source.

### BACKGROUND OF THE INVENTION

Mass spectrometry has been employed extensively for ion-ion chemistry experiments, in which analyte ions produced from a sample are reacted with reagent ions of opposite polarity. McLuckey et al. in “Ion/Ion Chemistry of High-Mass Multiply Charged Ions,” Mass Spectrometry Reviews, Vol. 17, pp. 369-407 (1998), discusses various examples of mass spectrometric studies of this type. It is known that reacting an appropriate reagent anion with a multiply charged analyte cation can result in a radical site being generated, which induces dissociation of the analyte cation into product ions. This process, which is known as electron transfer dissociation (ETD), is described by Hunt et al. in U.S. Pat. No. 7,534,622 entitled “Electron Transfer Dissociation for Biopolymer Sequence Mass Spectrometric Analysis,” as well as by Syka et al. in “Peptide and Protein Sequence Analysis by Electron Transfer Dissociation Mass Spectrometry,” Proc. Nat. Acad. Sci., vol. 101, no. 26, pp. 9528-9533 (2004), the contents of both of which are incorporated herein by reference. ETD is a particularly useful tool for proteomics research, since it yields information complementary to that obtained by conventional dissociation techniques (e.g., collisionally induced dissociation), and also because ETD tends to generate product ions having intact post-translational modifications.

Implementation of ETD or other ion-ion experiments in a mass spectrometer requires two ion sources: a first ion source for generating analyte ions from a sample, and a second ion source for generating reagent ions. Typically, the analyte ion source utilizes an ionization technique, such as electrospray ionization, that operates at atmospheric pressure. Atmospheric or near-atmospheric pressure ionization techniques have also been employed or proposed for production of reagent ions (e.g., Wells et al. “‘Dueling’ ESI: Instrumentation to Study Ion/Ion Reactions of Electrospray-Generated Cations and Anions,” J. Am. Soc. Mass Spectrometry, vol. 13, pp. 614-622 (2002), and U.S. Patent Application Publication No. 2008/0245963 by Land et al. entitled “Method and Apparatus for Generation of Reagent Ions in a Mass Spectrometer”). However, it has been found that atmospheric-pressure ionization techniques may not be well suited to production of certain labile ETD reagent ion species, which tend to be neutralized within the environment of an atmospheric-pressure ionization chamber via loss of electrons to background gas molecules, or which tend to form ion species that are unsuitable for ETD due to reaction with species that are present in the background gas.

Generation of reagent ions using a conventional chemical ionization (CI) technique has also been disclosed in the prior art (see, e.g., the aforementioned Syka et al. paper as well as U.S. Pat. No. 7,456,397 by Hartmer et al.), and has been implemented in at least one commercially available ion trap mass spectrometer. In such sources, reagent ions are formed by reaction of reagent vapor molecules with secondary electrons. CI sources typically employ an energized filament to produce a stream of electrons that preferentially ionizes secondary molecules. Reagent ions formed in the CI source may be directed through a dedicated set of ion optics, and introduced into a two-dimensional ion trap for reaction with analyte ions via an end of the trap opposite to the end through which the analyte ions are introduced, as described by Syka et al. Alternatively, analyte and reagent ions may be sequentially passed into a common aperture or end of an ion trap by an ion switching structure, as described in the Hartmer et al. patent.

Although mass spectrometer configurations utilizing a CI reagent ion source have been used successfully for ETD experiments, they are known to present a number of operational and design problems. The filaments in the CI source may fail in an unpredictable manner and need to be replaced frequently. Cleaning and maintenance of the CI source may require venting of the mass spectrometer and consequent downtime. Further, the need to provide dedicated guides or switching optics to direct ions from the CI source to the ion trap complicates instrument design and may interfere with the ability to incorporate additional components into the ion path, such as e.g. other mass analyzers.

Shabanowitz et al. describe another reagent ion source for a mass spectrometer in United States Patent Application Publication No. 2009/0294649, entitled “Method and Apparatus for Generation of Reagent Ions in a Mass Spectrometer,” the entire contents of which are incorporated herein by reference. The reagent ion source has a source of reagent vapor that supplies gas-phase reagent molecules to a reagent ionization volume maintained at low vacuum pressure. The reagent vapor is generated by heating a quantity of the reagent substance in condensed-phase form, and is transported to the reagent ionization volume by entrainment in a carrier gas stream. A voltage source applies a potential across electrodes that are disposed in the reagent ionization volume to produce an electrical discharge (e.g., a glow discharge), which ionizes the reagent vapor to generate reagent ions. The reagent ions flow through an outlet to a reduced-pressure chamber of the mass spectrometer, and are thereafter directed to an ion trap or other structure for reaction with oppositely charged analyte ions. In specific implementations, the reagent may take the form of a polyaromatic hydrocarbon suitable for use as an ETD reagent.

The reagent ions are selectively admitted and transported through downstream ion optics from the reduced-pressure chamber to the ion trap or other structure, such as by adjusting the polarities and amplitudes of the DC offset voltages applied to the ion optics. In this way the so-called ion injection time, during which time the reagent ions are provided into the ion trap or other mass spectrometer component, may be varied in a controllable fashion. During operation, an initial ion injection time for the reagent ions is set in order to achieve a desired reagent ion population. At intervals of time, a pre-scan is performed to measure the actual reagent ion population that is injected. As the system becomes contaminated over an extended period of operation, resulting in reduced ionization efficiency, the measured reagent ion population decreases. In order to compensate for the reduced ionization efficiency, a new ion injection time longer than the initial ion injection time for the reagent ions is set, such that the desired reagent ion flux is once again achieved. The process of periodically increasing the length of the ion injection time for the reagent ions continues until a maximum operating value, such as for instance about 50 ms, is reached. Of course, it is necessary to clean the reagent ion source when the maximum injection time is reached. Unfortunately, cleaning and maintenance of the reagent ion source may require venting of the mass spectrometer and consequent downtime.

It would be advantageous to provide a method that overcomes or mitigates at least some of the above-mentioned limitations of the prior art.

### SUMMARY OF EMBODIMENTS OF THE INVENTION

According to an aspect of at least one embodiment of the instant invention, there is provided a method of providing reagent ions to a mass spectrometer, comprising: i) delivering a reagent species to a reagent ionization volume via a passageway at a flow rate; ii) using previously acquired information, calculating an injection time duration for injecting reagent ions that are formed in the reagent ionization volume into a reaction region of the mass spectrometer; iii) determining whether the calculated injection time duration is within a specified range of injection time duration values; and, iv) adjusting the flow rate at which the reagent species is delivered to the ionization volume when the calculated injection time duration falls outside of the specified range of injection time duration values.

According to an aspect of at least one embodiment of the instant invention, there is provided a method of providing reagent ions to a mass spectrometer, comprising: delivering a reagent species to a reagent ionization volume via a passageway at a flow rate; using previously acquired first information, calculating a first injection time duration for injecting reagent ions that are formed in the reagent ionization volume into a reaction region of the mass spectrometer; determining whether the calculated first injection time duration is within a specified first range of injection time duration values; and, when it is determined that the calculated first injection duration time falls outside of the specified first range of injection time duration values, then: during a first period of time, adjusting the flow rate of reagent species that is provided to the reagent ionization volume; and, during a second period of time that is subsequent to the first period of time, specifying a second range of injection time duration values, the calculated first injection time duration falling within the specified second range of injection time duration values, wherein during the second period of time, the flow rate of reagent species that is provided to the reagent ionization volume is substantially a maximum flow rate of the reagent species.

According to an aspect of at least one embodiment of the instant invention, there is provided a method of providing reagent ions to a mass spectrometer, comprising: providing a source of a reagent species in fluid communication with a reagent ionization volume of the mass spectrometer, the source having at least one controllably variable parameter for adjusting a flux of the reagent species for delivery to the reagent ionization volume; during a first period of time, controllably varying the at least one parameter so as to increase the flux of the reagent species relative to the flux of the reagent species that is obtained absent varying the at least one parameter, and calculating an injection time duration that is less than a first specified maximum injection time duration for injecting approximately a predetermined reagent ion population, the calculating performed using information that is acquired subsequent to controllably varying the at least one parameter; and, during a second period of time that is subsequent to the first period of time, increasing the injection time duration for injecting approximately the predetermined reagent ion population gradually between the first specified maximum injection time duration and a second specified maximum injection time duration that is longer than the first specified maximum injection time duration; wherein an approximately uniform reagent ion population is injected into the reaction region of the mass spectrometer during each injection pulse of a sequence of injection pulses that occurs during the first period of time and during the second period of time.

According to an aspect of at least one embodiment of the instant invention, there is provided a reagent ion source for a mass spectrometer, comprising: a reagent ionization volume for receiving a reagent species in gaseous form and for ionizing the reagent species to provide reagent ions for introduction into a reaction region of the mass spectrometer; a reagent species delivery portion in fluid communication with the reagent ionization volume via a passageway, the reagent species delivery portion for providing the reagent species in gaseous form and at a controllably variable flow rate for delivery into the reagent ionization volume, the reagent species delivery portion having at least one adjustable parameter for controllably varying the flow rate of the reagent species in gaseous form; and, a controller for calculating a reagent ion injection time duration based on previously acquired information, for determining whether the calculated reagent ion injection time duration is within a specified first range of reagent ion injection time duration values, and for adjusting the flow rate of the gaseous reagent species when the calculated reagent ion injection time duration is determined to be outside the specified first range of reagent ion injection time duration values and when the flow rate of the gaseous reagent species is less than a specified maximum flow rate value.

According to an aspect of at least one embodiment of the instant invention, there is provided a controller for controlling a system for providing reagent ions to a mass spectrometer, comprising a processor for executing programming code for performing: calculating a number of reagent ions provided per unit of time; based on a result of the calculating, determining whether to i) vary an operating parameter of the system for increasing a flux of neutral reagent species into an ionization volume of the system, or ii) increase a duration of time for providing reagent ions from the ionization volume into the mass spectrometer; providing a first control signal for varying the operating parameter of the system when the determination is indicative of varying the operating parameter; and, providing a second control signal for increasing the duration of time for providing reagent ions when the determination is indicative of increasing the duration of time for providing reagent ions, wherein the first and second control signals are provided for a same system during different periods of time.

### BRIEF DESCRIPTION OF THE DRAWINGS

Exemplary embodiments of the invention will now be described in conjunction with the following drawings, in which:

FIG. 1 is a symbolic diagram of an ion trap mass spectrometer incorporating a front-end reagent ion source with ion population control, in accordance with an embodiment of the invention;

FIG. 2 is a symbolic diagram showing details of the reagent ionization volume of FIG. 1;

FIG. 3 is a symbolic diagram of an alternate mass spectrometer arrangement incorporating a reagent ion source with ion population control, in accordance with an embodiment of the invention;

FIG. 4 is a simplified flow diagram of a method according to an embodiment of the instant invention; and,

FIG. 5 is a simplified flow diagram of a method according to another embodiment of the instant invention.

### DETAILED DESCRIPTION OF EMBODIMENTS OF THE INVENTION

The following description is presented to enable a person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the scope of the invention. Thus, the present invention is not intended to be limited to the embodiments disclosed, but is to be accorded the widest scope consistent with the principles and features disclosed herein. Throughout the detailed description and in the appended claims, the term “injection time duration” is defined as the length of a period of time that is calculated for providing approximately a predetermined population of reagent ions for reacting with an analyte species, and is measured in time units such as for instance milliseconds (ms). The terms “reagent ion injection time (tᵢₙⱼ) range” and “range of injection time duration values” are defined as a range of “injection time durations,” between a minimum specified “injection time duration” and a maximum specified “injection time duration.”

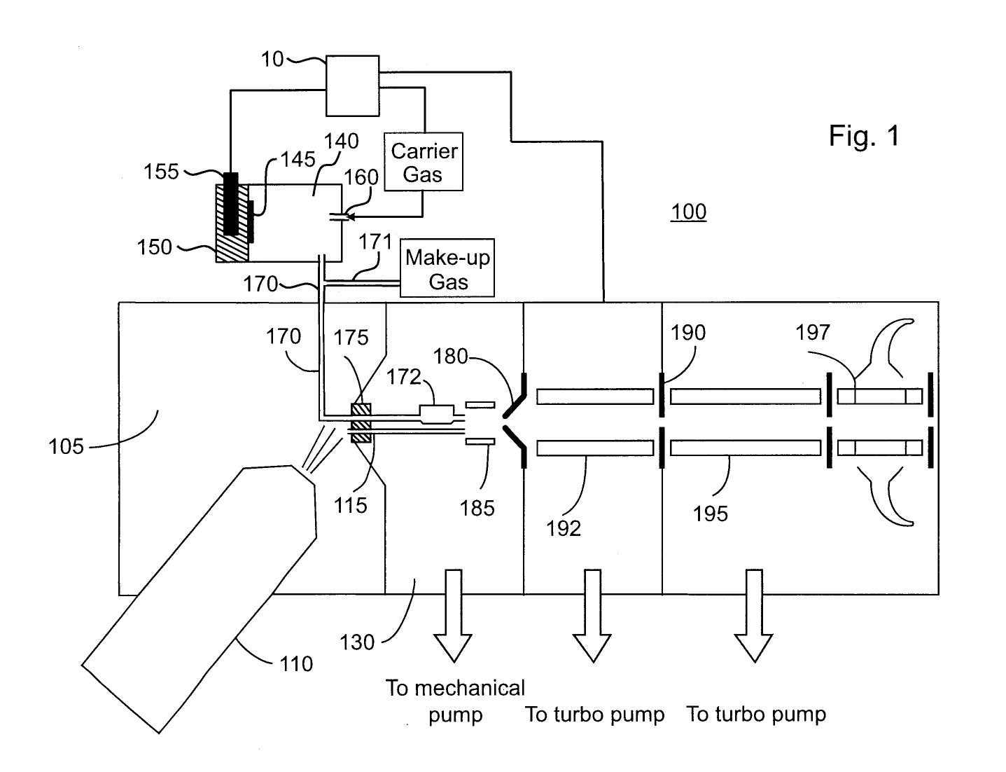

FIG. 1 schematically depicts a mass spectrometer 100 incorporating a front-end reagent ion source of the glow-discharge type. As used herein, the term “front-end” denotes that the ion source is configured to introduce reagent ions into a region located upstream in the analyte ion path relative to components of mass spectrometer 100 that are disposed in lower-pressure chambers (e.g., a mass analyzer), such that the analyte ions and reagent ions traverse a common path. Analyte ions (typically multiply-charged cations) are produced by electrospraying a sample solution into an analyte ionization chamber 105 via an electrospray probe 110. Analyte ionization chamber 105 is maintained typically at or near atmospheric pressure. The analyte ions, together with background gas and partially desolvated droplets, flow into the inlet end of a conventional ion transfer tube 115 (which may take the form of a narrow-bore capillary tube) and traverse the length of the tube under the influence of a pressure gradient. Analyte ion transfer tube 115 is preferably held in good thermal contact with a heated block (not depicted). As is known in the art, heating of the ion/gas stream passing through analyte ion transfer tube 115 assists in the evaporation of residual solvent and increases the number of analyte ions available for measurement. The analyte ions emerge from the outlet end of analyte ion transfer tube 115, which opens to reduced-pressure chamber 130. As indicated by the arrow, chamber 130 is evacuated to a low vacuum pressure (typically within the range of 0.1-50 Torr, and more typically between 0.5 and 10 Torr) by a mechanical pump or equivalent.

To produce reagent vapor for production of the requisite reagent ions, having a polarity opposite to that of the analyte ions, a reagent evaporation chamber 140 is provided having located therein a volume of a reagent substance 145 in condensed-phase (solid or liquid) form. By way of a few specific and non-limiting examples, a polyaromatic such as fluoranthene for ETD reagent ions, or benzoic acid for proton transfer reaction (PTR) reagent ions, is provided. More particularly, reagent substance 145 is placed in thermal contact with a block 150, which is heated by a cartridge heater 155. Controlling the reagent vapor pressure within evaporation chamber 140 is accomplished by varying the temperature (via adjusting the power that is supplied to heater 155) of block 150. Optionally, the block 150 is also in thermal contact with a peltier device or another suitable cooling system (such as for instance channels within block 150 for circulating a cooling fluid therethrough), which permits controlled cooling of the block 150 in addition to heating, and thereby supporting the use of a wider variety of reagent species.

During operation a flow of a generally inert carrier gas (such as for instance one of nitrogen, argon or helium) is introduced at a controlled rate through inlet 160 opening to the interior of chamber 140 to assist in the transport of reagent vapor molecules. The carrier gas also functions to continuously purge the interior of chamber 140 to prevent the influx of oxygen or other reactive gas species, which can react with and destroy ions formed from the reagent vapor.

Molecules of the reagent vapor that are entrained in the carrier gas enter an inlet end of reagent transfer tube 170, and are mixed with a make-up gas that is introduced via make-up gas inlet 171. The purpose of the make-up gas is to control the pressure in reagent ionization volume 172. The molecules of the reagent species traverse the length of the tube 170 under the influence of a pressure gradient, and enter the reagent ionization volume 172. Reagent transfer tube 170 may be a narrow-bore capillary tube fabricated from a suitable material, which extends between the interior of reagent evaporation chamber 140 and the reagent ionization volume 172. Reagent transfer tube 170, or a portion thereof, may be heated to prevent condensation of reagent material on the inner surfaces of the tube walls.

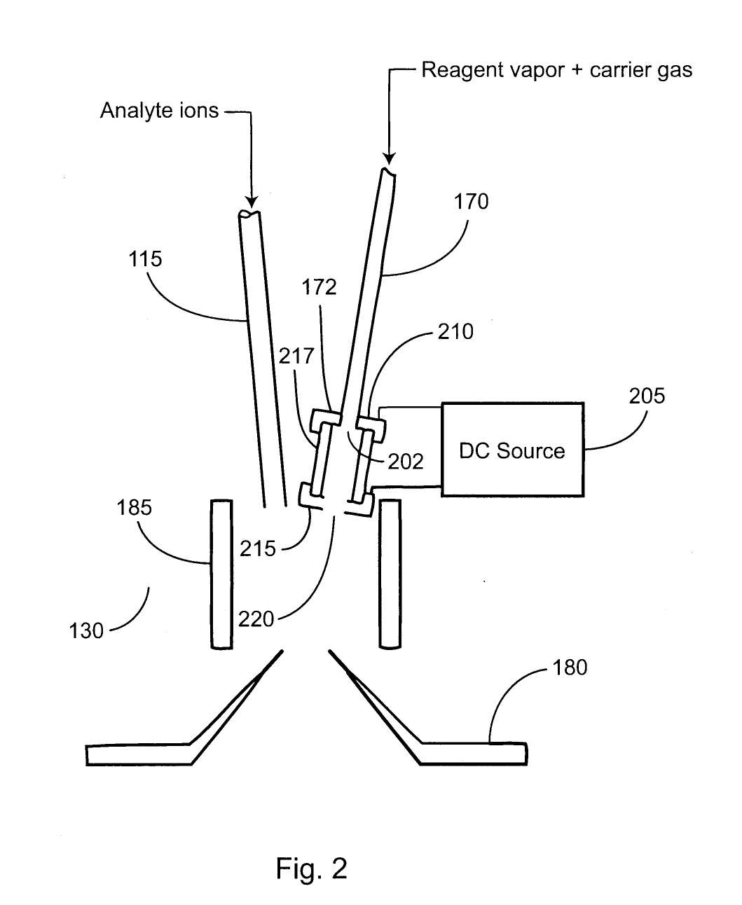

FIG. 2 shows an enlarged simplified view of a specific and non-limiting configuration of the reagent ionization volume 172. Other suitable configurations of the reagent ionization volume 172 are described in the previously mentioned United States Patent Application Publication No. 2009/0294649. In the specific and non-limiting configuration that is shown in FIG. 2, the reagent vapor enters reagent ionization volume 172 through an inlet 202 thereof. Reagent ionization volume 172 is located within chamber 130 of mass spectrometer 100, and functions to ionize (either directly or via a process involving intermediates) at least a portion of the reagent vapor transported thereto in order to produce the desired reagent ions (e.g., fluoranthene anions). For this purpose, reagent ionization volume 172 is provided with electrodes 210 and 215, across which a potential is applied by a voltage source 205 to establish a controlled discharge. In an embodiment the controlled discharge takes the form of a low-current (e.g., 1-100 μamp) discharge, such as a Townsend (dark) or glow discharge. As used herein, the term “reagent ionization volume” denotes a structure operable to effect ionization of the reagent vapor, and includes (without limitation) a structure having separated regions in which electrical discharge and ionization take place. Insulating sidewalls 217 extend between electrodes 210 and 215 and form with the electrodes a region that is generally closed to the exterior regions of chamber 130. Voltage source 205 will preferably include a current limiting circuitry to prevent transition of the low-current (e.g., glow) discharge to a high-current arc discharge.

Ionization volume 172 communicates with the interior volume of chamber 130 via a short outlet section or aperture 220, and is thus maintained at a sub-atmospheric pressure. The actual pressure within reagent ionization volume 172 is a function of the pressure maintained within chamber 130, the conductance of outlet section 220, and the flow rate of carrier gas/reagent vapor/make-up gas into ionization volume 172. Typically, the reagent ionization volume is operated to maintain the region at which the electrical discharge occurs at a pressure of between 0.5-10 Torr, although certain implementations may utilize pressures as low as 0.1 Torr or as high as 50 Torr.

Now referring to FIGS. 1 and 2, reagent ions are produced within ionization volume 172 by the direct or indirect interaction of reagent vapor molecules with electrons produced by the electrical discharge. The reagent ions exit ionization volume 172 through outlet section 220 and flow into chamber 130 under the influence of a pressure and/or electrical field gradient. The reagent ions may then be focused by tube lens 185 before passing into the succeeding chamber of mass spectrometer through an aperture in skimmer lens 180. It will be recognized that the analyte ions and reagent ions traverse a common path through the various ion transport optics (tube lens 185, skimmer lens 180, plate lens 190, and RF multipole ion guides 192 and 195) between chamber 130 and the reaction region, which may take the form of a two-dimensional quadrupole ion trap mass analyzer 197, as depicted in FIG. 1.

The analyte and reagent ion sources may be operated to provide a continuous supply of analyte and reagent ions into chamber 130. For ETD, the analyte and reagent ions are injected sequentially into a reaction region (e.g., ion trap 197). Selection of the ions to be delivered to ion trap 197 (i.e., the analyte or reagent ions) may be accomplished by applying DC voltages of suitable magnitude and polarity to the various ion transport optics, such that only the analyte ions are delivered to ion trap 197 at a first set of applied DC voltages, and only the reagent ions are delivered at a second set of DC voltages. Accordingly, the reagent ion injection time duration may be varied based on the length of time the second set of DC voltages are applied to the various ion transport optics.

It should be noted that the reaction region in which reaction of the analyte ions and reagent ions takes place may be located outside of the mass analyzer (e.g., in an intermediate multipole ion trap or ion guide), with subsequent conveyance of the product ions, or ions derived therefrom, to the mass analyzer for acquisition of a mass spectrum.

Referring still to FIG. 1, a controller 10 is provided for adjusting one or more operating parameters of the mass spectrometer system. In particular, the controller 10 is for adjusting a temperature of the reagent evaporation chamber 140 via cartridge heater 155 and block 145 (and optionally via a not illustrated Peltier device or other suitable cooling system). The controller 10 also controls a flow rate of the carrier gas that is introduced into the reagent evaporation chamber 140 via inlet 160. In addition, the controller 10 controls the DC voltages that are applied to the various ion transport optics, and thus the reagent ion injection time. During operation, a combination of the temperature of the reagent evaporation chamber 140, the carrier gas flow rate and the reagent ion injection time is determined for providing approximately a target number of ions to the reaction region of mass spectrometer 100 during a particular reagent ion injection event. The target number of reagent ions to be supplied to the reaction region is predetermined based on factors such as the ion storage capacity within the reaction region, expected analyte ion population, and desired reaction rate/efficiency. The controller 10 then adjusts the operating parameters of the mass spectrometer system, prior to performance of the reagent ion injection event. Additional adjustments to the operating parameters of the mass spectrometer system are performed either at regular or irregular intervals of time, or in response to a predetermined condition being satisfied, such as for instance when it is determined that the number of reagent ions injected is substantially smaller than the target number of reagent ions.

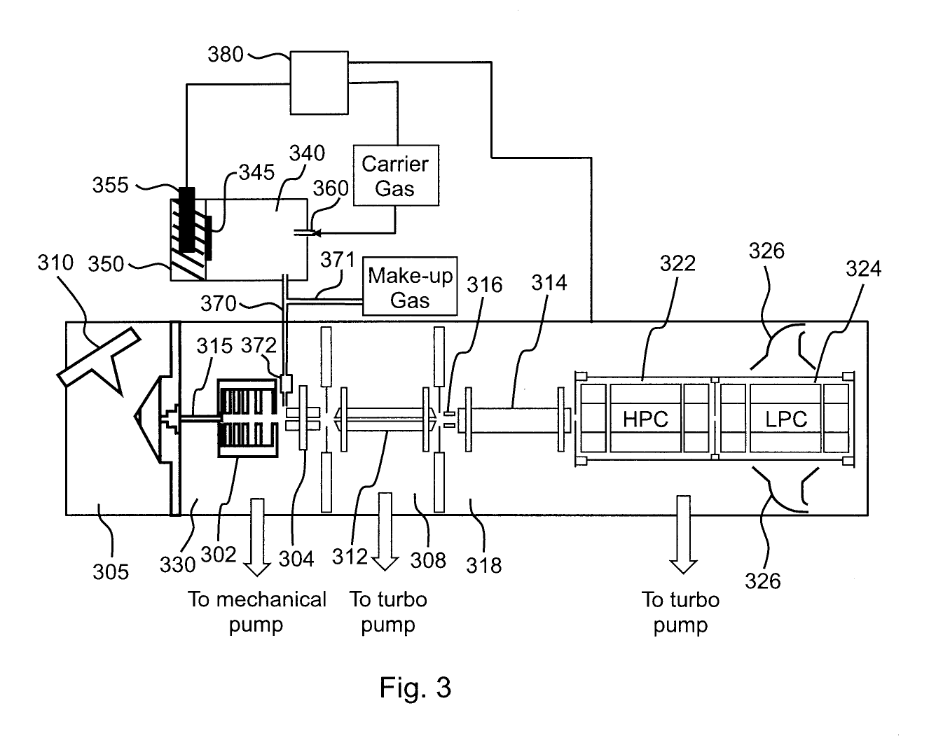

FIG. 3 is a simplified schematic diagram showing an alternate mass spectrometer system 300, which also incorporates a reagent ion source of the glow-discharge type. Analyte ions (typically multiply-charged cations) are produced by electrospraying a sample solution into an analyte ionization chamber 305 via an electrospray probe 310. Analyte ionization chamber 305 is maintained typically at or near atmospheric pressure. The analyte ions, together with background gas and partially desolvated droplets, flow into the inlet end of an ion transfer tube 315 (which may take the form of a narrow-bore capillary tube) and traverse the length of the tube 315 under the influence of a pressure gradient. Analyte ion transfer tube 315 is preferably held in good thermal contact with a heated block (not depicted). As is known in the art, heating of the ion/gas stream passing through analyte ion transfer tube 315 assists in the evaporation of residual solvent and increases the number of analyte ions available for measurement. The analyte ions emerge from the outlet end of analyte ion transfer tube 315, which opens to reduced-pressure chamber 330. As indicated by the arrow, chamber 330 is evacuated to a pressure typically within the range of 0.1-50 Torr, and more typically within the range of 0.5-10 Torr, by a mechanical pump or equivalent.

The analyte ions are then transmitted through radio frequency (RF) S-lens 302, which replaces the tube lens 180 and skimmer lens 185 of system 100. The S-lens 302 is a radio frequency (RF) only device that efficiently captures and focuses the ions into a tight beam without needing a DC gradient to propel the ions forward. The S-lens 302 includes a plurality of flat ring electrodes to which RF voltages are applied, with opposite phases applied to even-numbered and odd-numbered electrodes. Orifice diameters of the ring electrodes at the entrance of the S-lens 302 are larger, in order to capture as many of the ions emanating from the ion transfer tube 315 as possible. Orifice diameters of the ring electrodes in the rest of the S-lens 302 are smaller so as to focus the ion beam in a radially inward direction. Starting at the entrance, spacing between each ring electrode increases. The S-lens 302 generates confining RF electric fields that focus the ion beam as it travels through the device, thereby significantly increasing transmission of ions into the downstream components of the mass spectrometer system 300. The design and operation of an S-lens are discussed in greater detail in U.S. Patent Application Publication No. US2009/0045062A1 by Senko et al. (incorporated herein by reference).

Referring still to FIG. 3, the reagent ion source is configured to introduce reagent ions into a region of mass spectrometer 300 that is disposed between the exit of the S-lens 302 and a first RF-only quadrupole 304. To produce reagent vapor for production of the requisite reagent ions, having a polarity opposite to that of the analyte ions, a reagent evaporation chamber 340 is provided having located therein a volume of a reagent substance 345 in condensed-phase (solid or liquid) form. By way of a few specific and non-limiting examples, a polyaromatic such as fluoranthene for ETD reagent ions, or benzoic acid for proton transfer reaction (PTR) reagent ions, is provided. More particularly, reagent substance 345 is placed in thermal contact with a block 350, which is heated by a cartridge heater 355. Controlling the reagent vapor pressure within evaporation chamber 340 is accomplished by varying the temperature (via adjusting the power that is supplied to heater 355) of block 350. Optionally, the block 350 is also in thermal contact with a Peltier device or another suitable cooling system (such as for instance channels within block 350 for circulating a cooling fluid therethrough), which permits controlled cooling of the block 350 in addition to heating, and thereby supporting the use of a wider variety of reagent species.

During operation a flow of a generally inert carrier gas (such as for instance one of nitrogen, argon or helium) is introduced at a controlled rate through inlet 360 opening to the interior of chamber 340 to assist in the transport of reagent vapor molecules. The carrier gas also functions to continuously purge the interior of chamber 340 to prevent the influx of oxygen or other reactive gas species, which can react with and destroy ions formed from the reagent vapor.

Molecules of the reagent vapor that are entrained in the carrier gas enter an inlet end of reagent transfer tube 370, and are mixed with a make-up gas that is introduced via make-up gas inlet 371. The purpose of the make-up gas is to control the pressure in reagent ionization volume 372. The molecules of the reagent species traverse the length of the tube 370 under the influence of a pressure gradient. Reagent transfer tube 370, or a portion thereof, optionally is heated to prevent condensation of reagent material on the inner surfaces of the tube walls. By way of a specific and non-limiting example, reagent transfer tube 370 is a narrow-bore capillary tube fabricated from a suitable material, which extends between the interior of reagent evaporation chamber 340 and reagent ionization volume 372. Reagent ionization volume 372 may be substantially similar to the reagent ionization volume 172, which is described with reference to FIG. 2. Reagent ionization volume 372 operates in a manner that is analogous to the operation of reagent ionization volume 172, as discussed previously with reference to FIG. 2. The reagent ions that are produced within reagent ionization volume 372 and the analyte ions emerging from the S-lens 302 propagate together to the first RF only quadrupole 304.

In one operating mode, reagent ions that are formed in the reagent ionization volume 372 enter the first RF only quadrupole 304, propagate through quadrupole 312 in chamber 308 to octapole 314, via split gate lens 316 disposed in chamber 318, and finally arrive at a mass analyzer, such as for instance the first cell 322 of a dual-pressure linear ion trap disposed within chamber 318. An ETD or PTR reaction between analyte ions and the reagent ions occurs within the first cell 322 of the ion trap mass analyzer, and the product ions of the ETD or PTR reaction are mass analyzed using the second cell 324. In the instant and non-limiting example, the first cell 322 of the dual-pressure ion trap is held at a higher pressure (˜5×10⁻³ Torr) than in previous linear ion trap systems to improve efficiency of trapping, isolating, and fragmenting ions of interest. The second cell 324 is held at a lower pressure (˜4×10⁻⁴ Torr), to allow for faster mass analysis scans with increased resolution, as detected using detectors 326.

Referring still to FIG. 3, a controller 380 is provided for adjusting one or more operating parameters of the mass spectrometer system 300. In particular, the controller 380 is for adjusting a temperature of the reagent evaporation chamber 340 via cartridge heater 355 and block 345 (and optionally via a not illustrated Peltier device or other suitable cooling system). The controller 380 also controls a flow rate of the carrier gas that is introduced into the reagent evaporation chamber 340 via inlet 360. In addition, the controller 380 controls the DC voltages that are applied to the various ion transport optics, and thus controls the reagent ion injection time. During operation, a combination of the temperature of the reagent evaporation chamber 340, the carrier gas flow rate and the reagent ion injection time is determined for providing approximately a target number of ions to the reaction region of mass spectrometer 300 during a particular reagent ion injection event. The controller 380 then adjusts the operating parameters of the mass spectrometer system 300, prior to performance of the reagent ion injection event. Additional adjustments to the operating parameters of the mass spectrometer system are performed either at regular or irregular intervals of time, or in response to a predetermined condition being satisfied, such as for instance when it is determined that the number of reagent ions injected is substantially smaller than the target number of reagent ions.

Of course, other alternate mass spectrometer system arrangements may be implemented. Such systems may include, by way of specific and non-limiting examples, ion mobility cells or T-wave cells either in addition to or in place of some of the components of the systems that are described with reference to FIGS. 1-3. Optionally, the ETD or PTR reaction occurs within the ion mobility or T-wave cell, when such cells are present. Further optionally, the reagent ion source is arranged to deliver reagent ions via an end of the ion trap mass analyzer that is opposite the analyte ion introduction end.

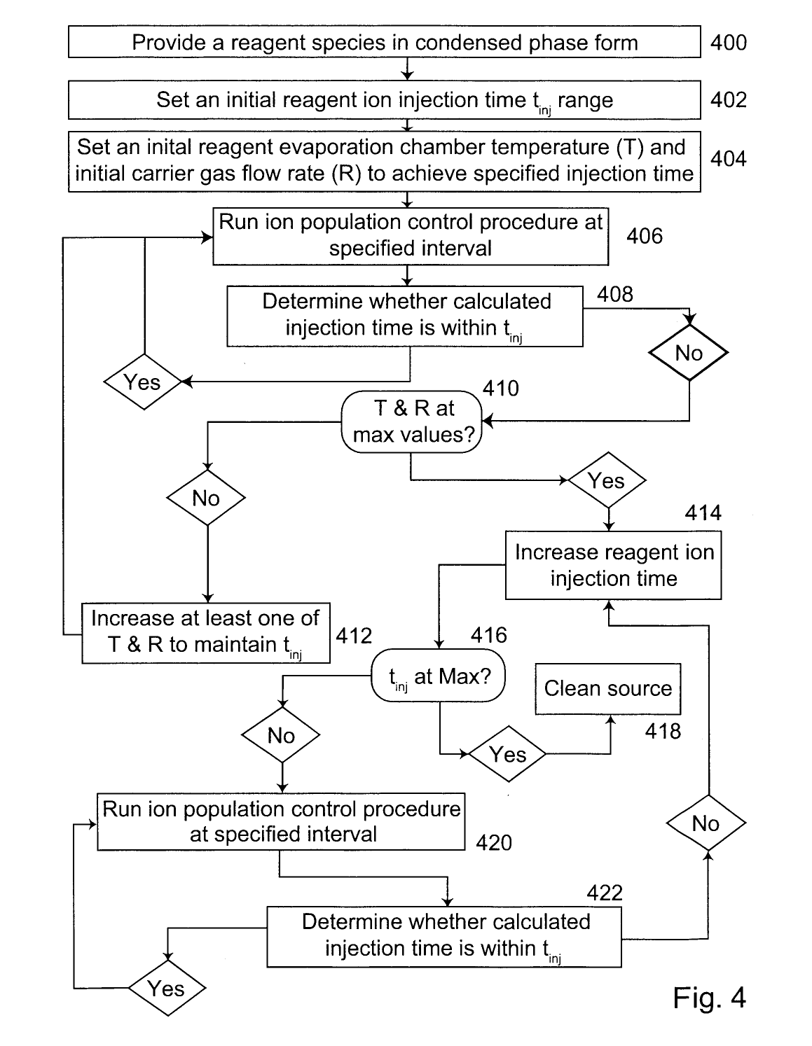

Referring now to FIG. 4, shown is a simplified flow diagram of a method according to an embodiment of the instant invention. In the method of FIG. 4, the reagent species is provided in condensed phase (solid or liquid) form. It should be noted that the method of FIG. 4 is described by way of a specific and non-limiting example in terms of the operation of the mass spectrometer system 100 of FIG. 1. That being said, the method of FIG. 4 may equivalently be used with the mass spectrometer system 300 that is shown in FIG. 3, or with another not illustrated mass spectrometer system having a suitable configuration. At 400 a reagent species 145 in condensed phase form (liquid or solid) is provided within reagent evaporation chamber 140 and in thermal communication with temperature control block 150. At 402 an initial reagent ion injection time (tᵢₙⱼ) range is set. By way of a specific and non-limiting example, the initial reagent ion injection time range is set to between 2 and 10 ms. At 404 an initial reagent evaporation chamber temperature (T) and an initial carrier gas flow rate (R) are set to achieve the injection of a desired reagent ion population using an injection time duration that is within the specified initial reagent ion injection time range. At 406 the controller 10 runs an ion population control procedure, at predetermined intervals of time. In particular, a pre-scan is performed to measure the number of reagent ions, or the reagent ion flux, that is injected into the instrument. Based on the pre-scan, the injection time duration that is necessary to obtain a desired (target) reagent ion population (number of reagent ions) is calculated. At 408, a determination is made as to whether or not the calculated injection time duration is within the initial reagent ion injection time range. If “yes,” then the analytical scan is performed using the calculated injection time duration. If “no,” then at 410 a determination is made as to whether the current values of T and R are maximum respective values. If “no,” then the controller 10 increases or increments at least one of T and R at 412, and returns to step 406 to resume the reagent ion population control procedure with the new T and/or R settings. If “yes,” then at 414 a new reagent ion injection time range is set, which is also referred to in the following steps as the current reagent ion injection time range. More particularly, each one of the new minimum and maximum injection time durations of the current reagent ion injection range is longer than the corresponding one of the minimum and maximum injection time durations of the initial reagent ion injection time range. By way of a specific and non-limiting example, the new reagent ion injection time range defines minimum and maximum injection time durations of 10 and 15 ms, respectively. At 416 it is determined whether the new reagent ion injection time range exceeds a maximum value, typically 40-50 ms. If “yes,” then at 418 the source is cleaned. If “no,” then at 420 the controller 10 runs an ion population control procedure, at predetermined intervals of time. As described above, the ion population control procedure comprises performing a pre-scan to measure the number of reagent ions, or the reagent ion injection rate, that is injected into the instrument. Based on the pre-scan, the injection time that is necessary to obtain the desired reagent ion population is calculated. At 422, a determination is made as to whether or not the calculated injection time is within the current reagent ion injection time range. If “yes,” then an analytical scan is performed using the calculated current reagent ion injection time. If “no,” then at 414 a new reagent ion injection time range is set. This procedure continues until it is determined at 416 that the reagent ion injection time range is set to the maximum allowable value thereof, and the source is cleaned at 418.

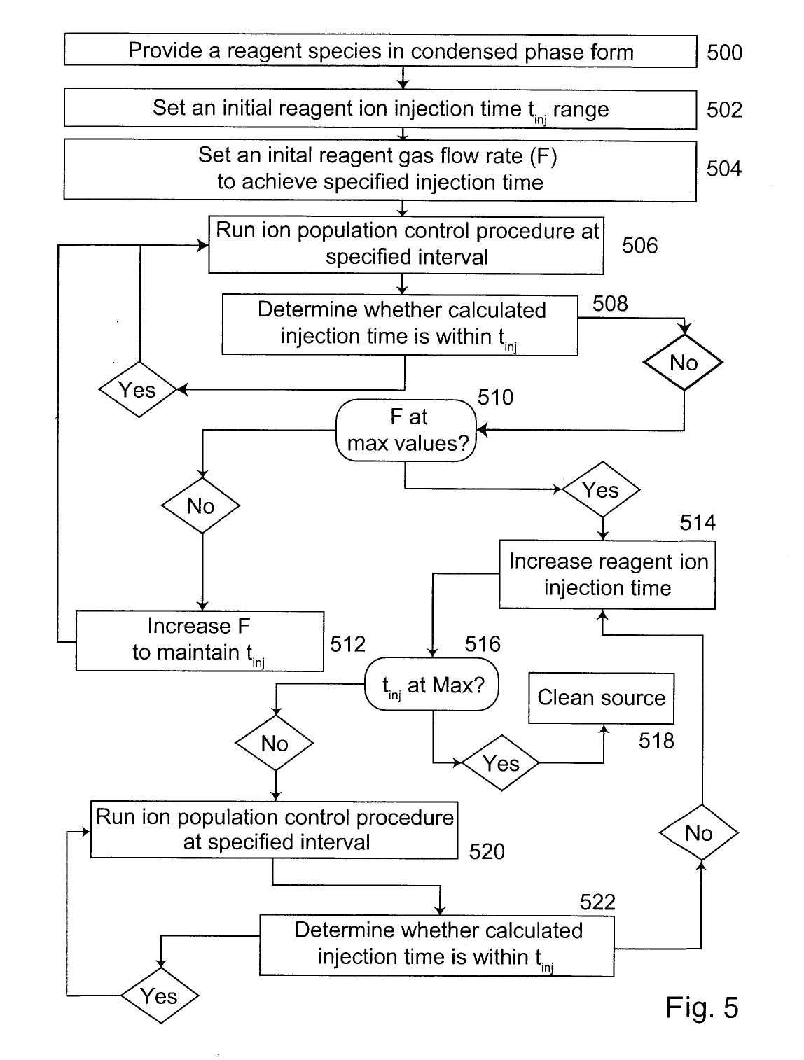

Referring now to FIG. 5, shown is a simplified flow diagram of a method according to an embodiment of the instant invention. In the method of FIG. 5, the reagent species is provided in gaseous phase (vapor) form. It should be noted that the method of FIG. 5 is described by way of a specific and non-limiting example in terms of the operation of a mass spectrometer system similar to the one that is shown in FIG. 1. Since the reagent species is provided in gaseous form, the evaporation chamber 140 is not necessary. Instead, the reagent vapor in gaseous phase form is provided from a not-illustrated source into the reagent transfer tube 170, where it is mixed with a make-up gas that is introduced via make-up gas inlet 171. The mixed reagent vapor and make-up gas is delivered to ionization volume 172 under the influence of a pressure gradient, as described previously with reference to FIG. 1. A not illustrated flow controller is used to set an initial flow rate of the reagent vapor that is provided from the source, and to vary the flow rate in response to control signals that are received from the controller 10. Of course, the method of FIG. 4 may equivalently be used with a mass spectrometer system similar to the one that is shown in FIG. 3, or with another not illustrated mass spectrometer system having a suitable configuration.

Referring still to FIG. 5, a reagent species in gaseous phase (vapor) form is provided at 500. At 502 an initial reagent ion injection time (tᵢₙⱼ) range is set. By way of a specific and non-limiting example, the initial reagent ion injection time range is set to between 2 and 10 ms. At 504, an initial reagent gas flow rate (F) is set to achieve the injection of a desired reagent ion population using an injection time duration that is within the specified initial reagent ion injection time range. At 506 the controller 10 runs an ion population control procedure, at predetermined intervals of time. In particular, a pre-scan is performed to measure the number of reagent ions, or the reagent ion flux, that is injected into the instrument. Based on the pre-scan, the injection time duration that is necessary to obtain a desired reagent ion population (number of reagent ions, or reagent ion flux) is calculated. At 508, a determination is made as to whether or not the calculated injection time duration is within the initial reagent ion injection time range. If “yes,” then an analytical scan is performed using the calculated injection time duration. If “no,” then at 510 a determination is made as to whether the current value of F is a maximum value. If “no,” then at 512 the controller 10 increases or increments the value of F, thereby increasing the reagent gas flow rate, and returns to step 506 to resume the reagent ion population control procedure using the new value of F. If “yes,” then at 514 a new reagent ion injection range is set, which is also referred to in the following steps as the current reagent ion injection range. More particularly, each one of the new minimum and maximum injection time durations of the current reagent ion injection range is longer than the corresponding one of the minimum and maximum injection time durations of the initial reagent ion injection range. By way of a specific and non-limiting example, the new reagent ion injection time range defines minimum and maximum injection time durations of 10 and 15 ms, respectively. At 516 it is determined whether the new reagent ion injection time range exceeds a maximum value, typically 40-50 ms. If “yes,” then at 518 the source is cleaned. If “no,” then at 520 the controller 10 runs an ion population control procedure, at predetermined intervals of time. As described above, the ion population control procedure comprises performing a pre-scan to measure the number of reagent ions, or the reagent ion injection rate, that is injected into the instrument. Based on the pre-scan, the injection time duration that is necessary to obtain the desired reagent ion population is calculated. At 522, a determination is made as to whether or not the calculated injection time duration is within the current reagent ion injection time range. If “yes,” then an analytical scan is performed using the calculated current reagent ion injection time duration. If “no,” then at 514 a new reagent ion injection time range is set. This procedure continues until it is determined at 516 that the reagent ion injection time range is set to the maximum allowable value thereof, and the source is cleaned at 518.

Optionally, the mass spectrometer system 100 that is shown in FIG. 1 or the mass spectrometer system 300 that is shown in FIG. 3 is modified to support the addition of an external standard for use during instrument calibration or as a lock mass. By way of a specific and non-limiting example, the external standard is contained within a second evaporation chamber communicating separately with the ionization volume 172 or 372. The temperature and carrier gas flow rate through the second chamber are controlled separately, in order to provide a desired population of the external standard ions.

It should be further recognized that the specific implementation depicted and described herein, i.e., where the reagent ion source takes the form of an ETD reagent ion source supplying ions to an analytical two-dimensional ion trap, are intended to be illustrative rather than limiting. A reagent ion source constructed in accordance with the invention may be beneficially utilized for supplying reagent ions of any suitable type and character to one or more reaction regions, which will not necessarily include a trapping structure.

Numerous other embodiments may be envisaged without departing from the scope of the instant invention.

What is claimed is:

1. A method of providing reagent ions to a mass spectrometer, comprising:
i) delivering a reagent species to a reagent ionization volume via a passageway at a flow rate;
ii) using previously acquired information, calculating an injection time duration for injecting reagent ions that are formed in the reagent ionization volume into a reaction region of the mass spectrometer;
iii) determining whether the calculated injection time duration is within a specified range of injection time duration values; and,
iv) adjusting the flow rate at which the reagent species is delivered to the ionization volume when the calculated injection time duration falls outside of the specified range of injection time duration values.

2. A method according to claim 1, wherein the previously acquired information is acquired during the performance of a prescan.

3. A method according to claim 2, wherein the previously acquired information is indicative of a number of reagent ions injected into the reaction region of the mass spectrometer during the performance of the prescan.

4. A method according to claim 1, comprising repeating i), ii) and iii) and prior to repeating iv), performing;
determining whether the flow rate is less than a maximum specified flow rate; and
when the calculated injection time duration falls outside of the specified range of injection time duration values and the flow rate is other than less than the maximum specified flow rate, specifying a different range of injection time duration values, the calculated injection time duration falling within the specified different range of injection time duration values.

5. A method according to claim 4, wherein the reagent species is generated by evaporation of a condensed phase volume contained within a reagent evaporation chamber, which chamber is in fluid communication with the reagent ionization volume via the passageway.

6. A method according to claim 5, wherein determining whether the flow rate is less than a maximum specified flow rate comprises determining whether at least one of:
an internal temperature of the reagent evaporation chamber is less than a specified maximum temperature; and,
a rate of carrier gas flow that is provided through the reagent evaporation chamber is less than a specified maximum rate of carrier gas flow.

7. A method according to claim 6, wherein adjusting the flow rate at which the reagent species is delivered to the ionization volume comprises adjusting the internal temperature of the reagent evaporation chamber.

8. A method according to claim 6, wherein adjusting the flow rate at which the reagent species is delivered to the ionization volume comprises adjusting the rate of carrier gas flow that is provided through the reagent evaporation chamber.

9. A method according to claim 5, wherein adjusting the flow rate at which the reagent species is delivered to the ionization volume comprises adjusting at least one of an internal temperature of the reagent evaporation chamber and a rate of carrier gas flow that is provided through the reagent evaporation chamber.

10. A method according to claim 4, wherein the reagent species is delivered via a flow controller that is in fluid communication with a source of the reagent species in the gaseous phase.

11. A method according to claim 1, comprising providing a source of an external standard separately in fluid communication with the ionization volume via the passageway, wherein the external standard is used for one of lockmass and calibration of the mass spectrometer.

12. A method of providing reagent ions to a mass spectrometer, comprising:
delivering a reagent species to a reagent ionization volume via a passageway at a flow rate;
using previously acquired first information, calculating a first injection time duration for injecting reagent ions that are formed in the reagent ionization volume into a reaction region of the mass spectrometer;
determining whether the calculated first injection time duration is within a specified first range of injection time duration values; and,
when it is determined that the calculated first injection time duration falls outside of the specified first range of injection time duration values, then:
during a first period of time, adjusting the flow rate of reagent species that is provided to the reagent ionization volume; and,
during a second period of time that is subsequent to the first period of time, specifying a second range of injection time duration values, the calculated first injection time duration falling within the specified second range of injection time duration values,
wherein during the second period of time, the flow rate of reagent species that is provided to the reagent ionization volume is substantially a maximum flow rate of the reagent species.

13. A method according to claim 12, comprising subsequent to adjusting the flow rate of reagent species to the reagent ionization volume, performing:
acquiring second information;
calculating using the second information a second injection time duration for injecting reagent ions that are formed in the reagent ionization volume into the reaction region of the mass spectrometer, the calculated second injection time duration falling within the specified first range of injection time duration values.

14. A method according to claim 13, wherein the previously acquired first information and the acquired second information are acquired during the performance of first and second prescans, respectively.

15. A method according to claim 12, wherein during the second period of time the flow rate is approximately constant.

16. A method according to claim 12, wherein the reagent species is generated by evaporation of a condensed phase volume contained within a reagent evaporation chamber, which chamber is in fluid communication with the reagent ionization volume via the passageway.

17. A method according to claim 16, wherein adjusting the flow rate of reagent species to the ionization volume comprises adjusting at least one of an internal temperature of the reagent evaporation chamber and a rate of carrier gas flow that is provided through the reagent evaporation chamber.

18. A method according to claim 12, wherein the reagent species is delivered via a flow controller that is in fluid communication with a source of the reagent species in the gaseous phase.

19. A method according to claim 12, comprising providing a source of an external standard separately in fluid communication with the ionization volume via the passageway, wherein the external standard is used for one of lockmass and calibration of the mass spectrometer.

20. A method of providing reagent ions to a mass spectrometer, comprising:
providing a source of a reagent species in fluid communication with a reagent ionization volume of the mass spectrometer, the source having at least one controllably variable parameter for adjusting a flux of the reagent species for delivery to the reagent ionization volume;
during a first period of time, controllably varying the at least one parameter so as to increase the flux of the reagent species relative to the flux of the reagent species that is obtained absent varying the at least one parameter, and calculating an injection time duration that is less than a first specified maximum injection time duration for injecting approximately a predetermined reagent ion population, the calculating performed using information that is acquired subsequent to controllably varying the at least one parameter; and,
during a second period of time that is subsequent to the first period of time, increasing the injection time duration for injecting approximately the predetermined reagent ion population gradually between the first specified maximum injection time duration and a second specified maximum injection time duration that is longer than the first specified maximum injection time duration;
wherein an approximately uniform reagent ion population is injected into the reaction region of the mass spectrometer during each injection pulse of a sequence of injection pulses that occurs during the first period of time and during the second period of time.

21. A reagent ion source for a mass spectrometer, comprising:
a reagent ionization volume for receiving a reagent species in gaseous form and for ionizing the reagent species to provide reagent ions for introduction into a reaction region of the mass spectrometer;
a reagent species delivery portion in fluid communication with the reagent ionization volume via a passageway, the reagent species delivery portion for providing the reagent species in gaseous form and at a controllably variable flow rate for delivery into the reagent ionization volume, the reagent species delivery portion having at least one adjustable parameter for controllably varying the flow rate of the reagent species in gaseous form; and,
a controller for calculating a reagent ion injection time duration based on previously acquired information, for determining whether the calculated reagent ion injection time duration is within a specified first range of reagent ion injection time duration values, and for adjusting the flow rate of the gaseous reagent species when the calculated reagent ion injection time duration is determined to be outside the specified first range of reagent ion injection time duration values and when the flow rate of the gaseous reagent species is less than a specified maximum flow rate value.

22. A reagent ion source for a mass spectrometer according to claim 21, wherein the reagent species delivery portion comprises an evaporation chamber for producing reagent species vapor from a source of the reagent species in condensed phase form.

23. A reagent ion source for a mass spectrometer according to claim 22, comprising a temperature control device in thermal communication with the evaporation chamber for controllably varying an internal temperature of the evaporation chamber, wherein the at least one adjustable parameter is the internal temperature of the evaporation chamber.

24. A reagent ion source for a mass spectrometer according to claim 23, wherein the temperature control device comprises a heater element.

25. A reagent ion source for a mass spectrometer according to claim 23, wherein the temperature control device comprises a Peltier device.

26. A reagent ion source for a mass spectrometer according to claim 23, wherein the temperature control device comprises a conduit in thermal communication with the evaporation chamber, the conduit for circulating a flow of a heat exchange fluid for transferring heat between the heat exchange fluid and the interior of the evaporation chamber.

27. A reagent ion source for a mass spectrometer according to claim 22, comprising a carrier gas inlet for receiving a flow of a carrier gas, wherein the at least one adjustable parameter is a rate of flow of the carrier gas into the evaporation chamber and out through the passageway.

28. A controller for controlling a system for providing reagent ions to a mass spectrometer, comprising a processor for executing programming code for performing:
calculating a number of reagent ions provided per unit of time;
based on a result of the calculating, determining whether to i) vary an operating parameter of the system for increasing a flux of neutral reagent species into an ionization volume of the system, or ii) increase a duration of time for providing reagent ions from the ionization volume into the mass spectrometer;
providing a first control signal for varying the operating parameter of the system when the determination is indicative of varying the operating parameter; and,
providing a second control signal for increasing the duration of time for providing reagent ions when the determination is indicative of increasing the duration of time for providing reagent ions,
wherein the first and second control signals are provided for a same system during different periods of time.
